(19) United States
(12) Patent Application Publication
(10) Pub. No.: US 2016/0015561 A1
(43) Pub. Date: Jan. 21, 2016

(54) Heatable and Coolable Therapeutic Device

(71) Applicant: Richard Leichter, Miller Place, NY (US)

(72) Inventor: Richard Leichter, Miller Place, NY (US)

(21) Appl. No.: 14/335,561

(22) Filed: Jul. 18, 2014

(51) Int. Cl. A61F 7/02

(52) U.S. Cl. CPC A61F 7/02; A61F 2007/0285; A61F 2007/0279; A61F 2007/0268

(57) ABSTRACT

A heatable and coolable therapeutic device provides a soothing sensation to areas of pain on a person's body. The device includes a first ball. The first ball is spaced from a second ball. Each of the first and second balls has a hollow interior. A spacer is coupled to and extends between the first ball and the second ball. The first ball, the second ball and the spacer define a cohesive unit. A heat-retentive material is positioned in the hollow interior of each of the first ball and the second ball. The heat-retentive material maintains a selectable one of an elevated temperature and a decreased temperature relative to ambient temperature over an extended period of time.

### BACKGROUND OF THE DISCLOSURE

### Field of the Disclosure

The disclosure relates to therapeutic devices and more particularly pertains to a new therapeutic device for providing a soothing sensation to areas of pain on a person's body.

### SUMMARY OF THE DISCLOSURE

An embodiment of the disclosure meets the needs presented above by generally comprising a first ball. The first ball is spaced from a second ball. Each of the first and second balls has a hollow interior. A spacer is coupled to and extends between the first ball and the second ball. The first ball, the second ball and the spacer define a cohesive unit. A heat-retentive material is positioned in the hollow interior of each of the first ball and the second ball. The heat-retentive material maintains a selectable one of an elevated temperature and a decreased temperature relative to ambient temperature over an extended period of time.

There has thus been outlined, rather broadly, the more important features of the disclosure in order that the detailed description thereof that follows may be better understood, and in order that the present contribution to the art may be better appreciated.

There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF THE DRAWINGS

The disclosure will be better understood and objects other than those set forth above will become apparent when consideration is given to the following detailed description thereof. Such description makes reference to the annexed drawings wherein:

FIG. 1 is a top front side perspective view of a heatable and coolable therapeutic device according to an embodiment of the disclosure showing the sleeve removed from the cohesive unit.

FIG. 2 is a top front side perspective view of an embodiment of the disclosure showing the cohesive unit positioned in and enveloped by the sleeve.

FIG. 3 is a front view of an embodiment of the disclosure.

FIG. 4 is a cross-sectional view of an embodiment of the disclosure taken along line 4-4 of FIG. 3.

FIG. 5 is a detailed side view of an embodiment of the disclosure.

FIG. 6 is an in-use top view of an embodiment of the disclosure.

### DESCRIPTION OF THE PREFERRED EMBODIMENT

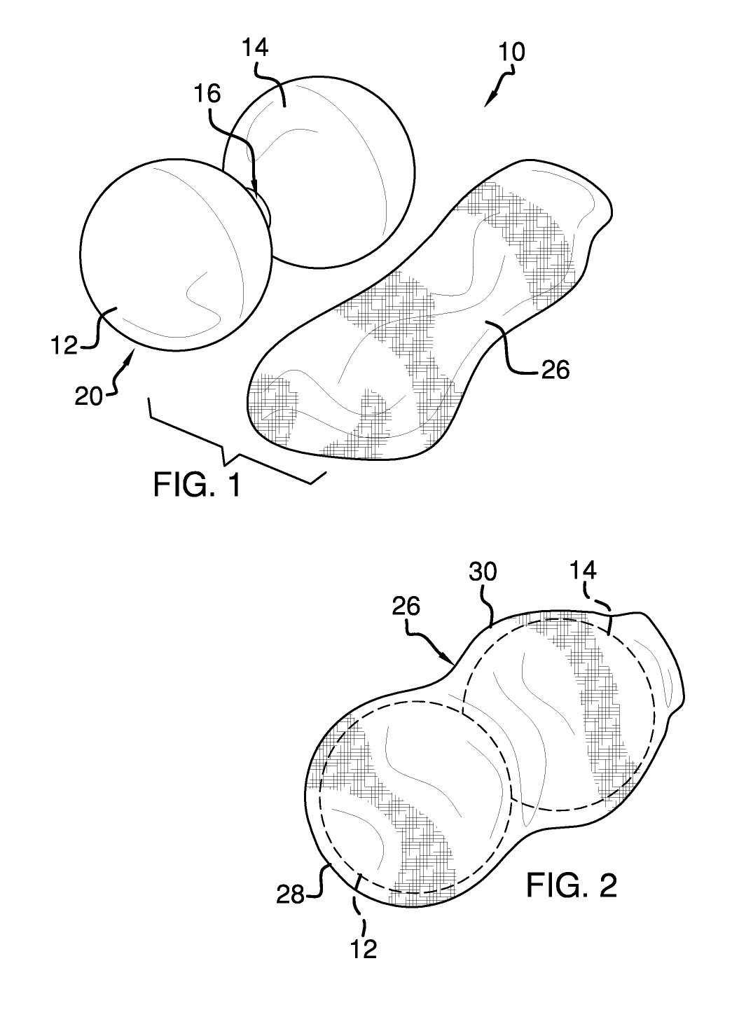

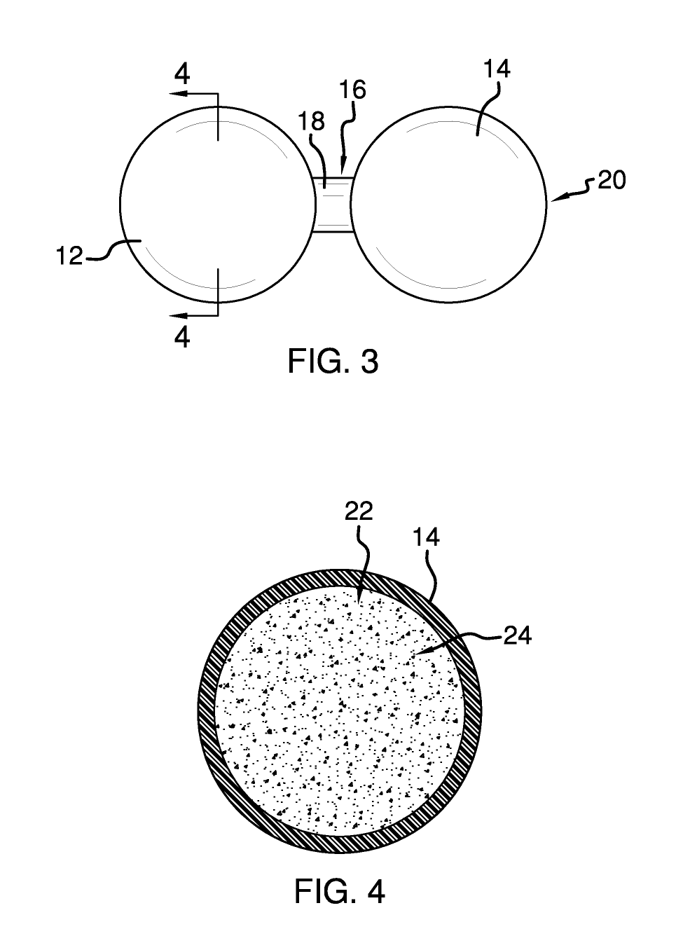

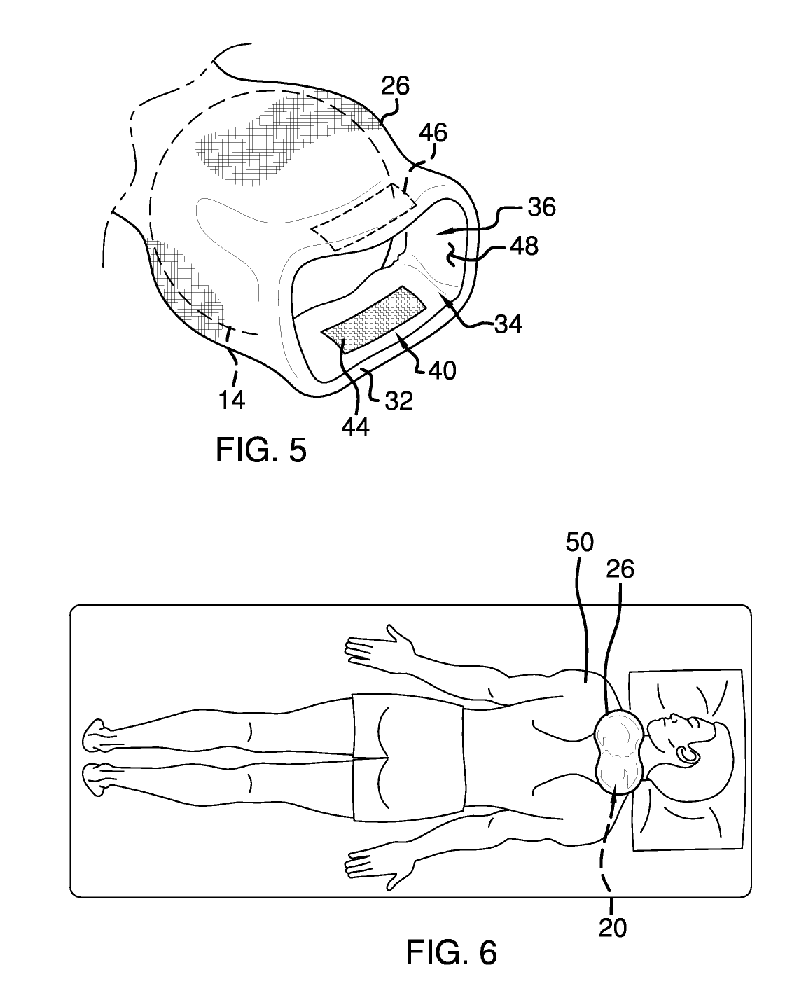

With reference now to the drawings, and in particular to FIGS. 1 through 6 thereof, a new therapeutic device embodying the principles and concepts of an embodiment of the disclosure and generally designated by the reference numeral 10 will be described.

As best illustrated in FIGS. 1 through 6, the heatable and coolable therapeutic device 10 generally comprises a first ball 12. A second ball 14 is provided and spaced from the first ball 12. Each of the first ball 12 and the second ball 14 may be constructed from a synthetic or natural polymer. The first ball 12 and the second ball 14 may each be spherical.

A spacer 16 is coupled to and extends between the first ball 12 and the second ball 14. The spacer 16 may comprise a tubular rod 18. Each of the first ball 12, the second ball 14 and the spacer 16 define a cohesive unit 20. The spacer 16 may have a length between the first ball 12 and the second ball 14 measuring between approximately 0.5 cm and 1.5 cm.

Each of the first ball 12 and the second ball 14 has a hollow interior 22. A heat-retentive material 24 is positioned in the hollow interior 22 of each of the first ball 12 and the second ball 14. The heat-retentive material 24 is configured for maintaining a selectable one of an elevated temperature and a decreased temperature relative to ambient temperature over an extended period of time. The heat-retentive material 24 may comprise a liquid or gel-like substance that can be heated in a microwave oven to achieve the elevated temperature or cooled in a freezer to achieve the decreased temperature. The heat-retentive material 24 may comprise the same material used in conventional cooling or heating packs, which allow the decreased and elevated temperatures, respectively, to be maintained for at least a couple of hours.

A flexible sleeve 26 is provided having a bottom wall 28 and a perimeter wall 30 coupled to and extending upwardly from the bottom wall 28. A top edge 32 of the sleeve 26 defines an opening 34 into an interior space 36 of the sleeve 26. The cohesive unit 20 is positionable in the interior space 36 of the sleeve 26 such that the cohesive unit 20 is enveloped by the sleeve 26. The sleeve 26 may be elasticized such that the sleeve 26 conforms to a shape of the cohesive unit 20 when the cohesive unit 20 is placed in the interior space 36 of the sleeve 26. The sleeve 26 is elongated along a longitudinal axis extending from the bottom wall 28 to the top edge 32 of the sleeve 26.

A closure 40 is coupled to the sleeve 26 for retaining the top edge 32 of the sleeve 26 in a closed position covering the opening 34. The closure 40 may comprise a hook and loop fastener, snaps, or the like. The closure 40 comprises a first mating member 44 and a second mating member 46 that may each coupled to an interior surface 48 of the perimeter wall 30 proximate the top edge 32 of the sleeve 26.

In use, the cohesive unit 20 is placed into a freezer if a user 50 wishes to have the heat-retentive material 24 achieve the decreased temperature. Alternatively, the cohesive unit 20 can be placed into a microwave oven and heated in order to have the heat-retentive material 24 achieve the elevated temperature. The cohesive unit 20 is then positioned in the sleeve 26 and placed onto an area of the user's 50 body experiencing pain for a selectable period of time. Thus, the device 10 may be placed onto the user's 50 head, neck, back, or the like. After use, the sleeve 26 may be removed from the cohesive unit 20 and washed as desired.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A heatable and coolable therapeutic device comprising:
a first ball;
a second ball spaced from said first ball, each of said first ball and said second ball having a hollow interior;
a spacer coupled to and extending between said first ball and said second ball, each of said first ball, said second ball and said spacer defining a cohesive unit; and
a heat-retentive material positioned in said hollow interior of each of said first ball and said second ball, said heat-retentive material being configured for maintaining a selectable one of an elevated temperature and a decreased temperature relative to ambient temperature over an extended period of time.

2. The device of claim 1, further comprising each of said first ball and said second ball being spherical.

3. The device of claim 1, further comprising a sleeve having a bottom wall and a perimeter wall coupled to and extending upwardly from said bottom wall, a top edge of said sleeve defining an opening into an interior space of said sleeve, said cohesive unit being positionable in said interior space of said sleeve such that said cohesive unit is enveloped by said sleeve.

4. The device of claim 3, further comprising a closure coupled to said sleeve for retaining said top edge of said sleeve in a closed position covering said opening.

5. The device of claim 4, further comprising said closure comprising a hook and loop fastener.

6. The device of claim 4, further comprising wherein said closure comprises a first mating member and a second mating member each coupled to an interior surface of said perimeter wall proximate said top edge of said sleeve.

7. The device of claim 3, further comprising said sleeve being elasticized such that said sleeve conforms to a shape of said cohesive unit when said cohesive unit is placed in said interior space of said sleeve.

8. The device of claim 3, further comprising said sleeve being elongated along a longitudinal axis extending from said bottom wall to said top edge of said sleeve.

9. The device of claim 3, further comprising said sleeve comprising an insulating material to maintain the temperature of said thermal material when said cohesive unit is positioned in said interior space of said sleeve.

10. The device of claim 1, further comprising wherein said spacer comprises a tubular rod.

11. A heatable and coolable therapeutic device comprising:
a first ball;
a second ball spaced from said first ball, each of said first ball and said second ball having a hollow interior, each of said first ball and said second ball being spherical;
a spacer coupled to and extending between said first ball and said second ball, said spacer comprising a tubular rod, each of said first ball, said second ball and said spacer defining a cohesive unit;
a heat-retentive material positioned in said hollow interior of each of said first ball and said second ball, said heat-retentive material being configured for maintaining a selectable one of an elevated temperature and a decreased temperature relative to ambient temperature over an extended period of time;
a sleeve having a bottom wall and a perimeter wall coupled to and extending upwardly from said bottom wall, a top edge of said sleeve defining an opening into an interior space of said sleeve, said cohesive unit being positionable in said interior space of said sleeve such that said cohesive unit is enveloped by said sleeve, said sleeve being elasticized such that said sleeve conforms to a shape of said cohesive unit when said cohesive unit is placed in said interior space of said sleeve, said sleeve being elongated along a longitudinal axis extending from said bottom wall to said top edge of said sleeve; and
a closure coupled to said sleeve for retaining said top edge of said sleeve in a closed position covering said opening, said closure comprising a hook and loop fastener, said closure comprising a first mating member and a second mating member each coupled to an interior surface of said perimeter wall proximate said top edge of said sleeve.
